(19) United States
(12) Patent Application Publication
(10) Pub. No.: US 2026/0151342 A1
(43) Pub. Date: Jun. 4, 2026

(54) Minitablet, Preparation Method Thereof, and Formulation

(71) Applicant: Beijing Vagary Pharmaceutical Technology Ltd., Beijing (CN)

(72) Inventors: Zong Chao NING, Beijing (CN); Bei ZHANG, Beijing (CN)

(21) Appl. No.: 18/706,573

(22) PCT Filed: Oct. 28, 2022
(86) PCT No.: PCT/CN2022/128331

§ 371 Date: Jul. 10, 2024

(30) Foreign Application Priority Data
Nov. 2, 2021 (CN) 202111288747.8

(51) Int. Cl. A61K 9/20; A61K 31/198; A61K 31/215; A61K 31/401; A61K 31/495; A61K 31/519; A61K 31/7048

(52) U.S. Cl. CPC A61K 9/2054; A61K 9/2018; A61K 9/205; A61K 9/2059; A61K 9/2095; A61K 31/198; A61K 31/215; A61K 31/401; A61K 31/495; A61K 31/519; A61K 31/7048

(57) ABSTRACT

The disclosure belongs to the technical field of pharmaceuticals, and specially relates to a minitablet, a preparation method thereof, and a formulation. The minitablet includes components of an active ingredient, a filler, and/or a disintegrant, and the minitablet has a diameter of 1 mm to 5 mm and a tablet weight of 1 mg to 50 mg. A number of the minitablet included in the formulation is single or multiple. A preparation method of the minitablet is further provided. The minitablet can realize direct administration in small doses, shows accurate dosage, is convenient to use, and improves medication compliance. The preparation method can effectively solve the problem of content uniformity while reducing the complexity and difficulty of the process, which is more suitable for industrial production.

### CROSS REFERENCE TO RELATED APPLICATION

The present application is a national stage application of International Patent Application No. PCT/CN2022/128331, filed on Oct. 28, 2022, which claims priority to the Chinese Patent Application No. 202111288747.8, filed with the China National Intellectual Property Administration (CNIPA) on Nov. 2, 2021, and entitled “MINITABLET, PREPARATION METHOD THEREOF, AND FORMULATION”. The disclosure of the two applications is incorporated herein by reference in its entirety.

### TECHNICAL FIELD

The present disclosure belongs to the technical field of pharmaceuticals, and specifically relates to a minitablet, a preparation method thereof, and a formulation.

### BACKGROUND

Due to the disadvantages of difficulty in swallowing and the inability to finely adjust a fixed dose, oral solid formulations cannot meet the delivery needs of special populations and high-value specialty drugs. As a new trend in solid dosage form design, the increasing maturity of minitablets may change the future of oral formulations, allowing them to occupy a place in the field of high-value pharmaceuticals.

Minitablets refer to tablets having a diameter of 1 mm to 5 mm and have the advantages such as easy swallowing, flexible dosage, and controllable drug release rate of traditional tablets, pellets, and liquid formulations. The minitablets not only are suitable for patients with dysphagia, but also can meet the clinical needs of customized medicines.

However, there are technical bottlenecks in the preparation process of minitablets. Due to the limitations of excipient performance, the proportion of active ingredients cannot be further increased. A higher proportion of active ingredients may face a problem of content uniformity. Moreover, the process has been greatly expanded in the prior art to avoid the performance defects of excipients, thus gradually increasing the complexity and difficulty of the process. At present, there is still no better way to overcome the bottleneck.

From the perspective of tableting technology, the compression of both minitablets and ordinary tablets actually depends on the quality of granulation, tablet filling control, and pressure control. The mass and volume of minitablets are much smaller than those of ordinary tablets, and thus the requirements are relatively higher in all aspects. Mainly, the compression of tablets with low weight and low pressure tests the operating accuracy of a moving machinery as well as the sensitivity and response speed of electrical components. In addition to the need for high-quality punching dies, the compression of minitablet also challenges the overall performance of a tablet press.

From the perspective of preparation technology, it is necessary to ensure that the material has the desirable fluidity, compressibility, and particle size distribution to ensure the uniformity and formability of the minitablets. Accordingly, there are higher requirements for various control indicators of raw materials and excipients.

The present disclosure aims to provide a minitablet and a preparation method thereof, which can significantly reduce the dosage of excipients in some drugs. The proportion of active ingredients can be increased to 40% to 80% according to the physical and chemical properties of different active ingredients, thereby avoiding the problem of content uniformity. Meanwhile, the use of excipients with better performance can reduce the complexity and difficulty of the process, thereby showing more advantages in the production.

Moreover, the minitablets have advantages that cannot be replaced by conventional tablets in clinical use. The minitablets can accurately control the dosage, thereby reducing the various risks caused by inaccurate dosages, and are small in size and can avoid the swallowing difficulties in children and the elderly.

### SUMMARY

In order to overcome the above technical problems, the present disclosure provides a minitablet, a preparation method thereof, and a formulation. The preparation method can solve the problem of content uniformity, while reducing the complexity and difficulty of the process, and is suitable for industrial promotion and application.

To achieve the above object, the present disclosure provides the following technical solutions:

A minitablet, including the following components: an active ingredient and/or an excipient, where the minitablet has a diameter of 1 mm to 5 mm and a tablet weight of 1 mg to 50 mg; and the excipient includes any one or more selected from the group consisting of a filler, a disintegrant, a binder, a lubricant, and a glidant.

In some embodiments, the minitablet has a diameter of 1 mm to 3 mm and a tablet weight of 2 mg to 30 mg.

In some embodiments, the active ingredient has a particle size D₉₀ of less than or equal to 250 μm, preferably less than or equal to 100 μm, and more preferably less than or equal to 20 μm.

In some embodiments, a mixture of the active ingredient and/or the excipient has a particle size D₉₀ of less than or equal to 350 μm, and preferably less than or equal to 150 μm.

In some embodiments, the filler includes any one or more selected from the group consisting of starch, pregelatinized starch, powdered sugar, lactose monohydrate, lactitol, sucrose, glucose, fructose, dextrin, cyclodextrin, powdered cellulose, microcrystalline cellulose, aerosil, mannitol, sorbitol, xylitol, erythritol, xylan, maltitol, glycine, calcium chloride, calcium sulfate, calcium carbonate, calcium phosphate, calcium hydrogen phosphate, calcium glycerophosphate, calcium carboxymethylcellulose, sodium chloride, sodium starch phosphate, aluminum chloride, aluminum hydroxide, aluminum silicate, calcium aluminum silicate, and magnesium carbonate;
the disintegrant includes any one or more selected from the group consisting of starch, pregelatinized starch, polacrilin potassium, soybean polysaccharide, microcrystalline cellulose, sodium bicarbonate, potassium bicarbonate, potassium carbonate, citric acid, alginic acid, sodium alginate, sodium starch glycolate, methylcellulose, low-substituted hydroxypropyl cellulose, crospovidone, croscarmellose sodium, carboxymethylcellulose sodium, and carboxymethylcellulose calcium;
the binder includes any one or more selected from the group consisting of gelatin, acacia, xanthan gum, tragacanth, polyethylene glycol, pregelatinized starch, polyvinyl alcohol, starch, dextrin, chitin, lactose monohydrate, sucrose, chitosan, glucose, dextran, copovidone, polyvinylpyrrolidone, carbomer, hydroxypropyl cellulose, methylcellulose, ethylcellulose, ethyl methylcellulose, hypromellose, low-substituted hypromellose, and carboxymethylcellulose sodium; and
the lubricant and/or the glidant includes any one or more selected from the group consisting of silica, aerosil, talc, glyceryl behenate, glyceryl monostearate, glyceryl distearate, sucrose fatty acid ester, sucrose monolaurate, sucrose monopalmitate, polyethylene glycol, sodium laurylsulfate, docusate sodium, sodium palmitate, magnesium silicate, magnesium aluminum silicate, stearic acid, sodium stearate, calcium stearate, zinc stearate, magnesium stearate, and sodium stearyl fumarate.

In some embodiments, the disintegrant is a mixture of a polysaccharide and the alginic acid.

In some embodiments, a mass ratio of the polysaccharide to the alginic acid in the disintegrant is in a range of (2-6):(1-3).

In some embodiments, the polysaccharide includes any one or more selected from the group consisting of fucose, mannose, soybean polysaccharide, black soybean polysaccharide, tremella polysaccharide, galactan, and glucuronic acid.

In some embodiments, the disintegrant is a mixture of soybean polysaccharide, glucuronic acid, and alginic acid, where a mass ratio of the soybean polysaccharide, the glucuronic acid, and the alginic acid is in a range of (1-5):1:(1-3).

In some embodiments, the filler is a mixture of microcrystalline cellulose, erythritol, xylan, and starch.

In some embodiments, a mass ratio of the microcrystalline cellulose, the erythritol, the xylan, and the starch in the filler is in a range of (3-6):1:(1-2):(2-5).

In some embodiments, the starch includes any one or more selected from the group consisting of wheat starch, sweet potato starch, lotus root starch, corn starch, and tapioca.

In some embodiments, the disintegrant is a mixture of croscarmellose sodium and low-substituted hydroxypropyl cellulose and/or a mixture of sodium starch glycolate and pregelatinized starch.

In some embodiments, a mass ratio of the croscarmellose sodium to the low-substituted hydroxypropyl cellulose in the mixture is in a range of (1-4):1, and preferably 2:1; and a mass ratio of the sodium starch glycolate to the pregelatinized starch in the mixture is in a range of (1-4):1, and preferably 2:1.

In some embodiments, the filler is a mixture of microcrystalline cellulose and mannitol and/or a mixture of the microcrystalline cellulose and lactose monohydrate.

In some embodiments, a mass ratio of the microcrystalline cellulose to the mannitol in the mixture is in a range of (1-4):1, and preferably 2:1; and a mass ratio of the microcrystalline cellulose to the lactose monohydrate in the mixture is in a range of (1-4):1, and preferably 2:1.

In some embodiments, the minitablet includes the filler and the disintegrant.

In some embodiments, a mass ratio of the filler to the disintegrant is in a range of (1-2):(2-5).

In some embodiments, the minitablet includes the following components in parts by weight: 0.03 parts to 80 parts of the active ingredient, 20 parts to 30 parts of the filler, and 3 parts to 8 parts of the disintegrant.

In some embodiments, the minitablet includes the following components in parts by weight: 0.03 parts to 50 parts of the active ingredient, 20 parts to 30 parts of the filler, and 3 parts to 8 parts of the disintegrant.

In some embodiments, the active ingredient is any one or more selected from the group consisting of: captopril, indapamide, propranolol hydrochloride, bisoprolol fumarate, atenolol, bumetanide, furosemide, torasemide, minoxidil, bendroflumethiazide, hydrochlorothiazide, reserpine, digoxin, medigoxin, nifedipine, isosorbide dinitrate, isosorbide mononitrate, pentaerythritol tetranitrate, nitroglycerin, clonidine hydrochloride, warfarin sodium, spironolactone, lofexidine hydrochloride, glyburide, glimepiride, glipizide, voglibose, acarbose, clopidogrel bisulfate, aspirin, scopolamine butylbromide, anisodamine hydrobromide, metoclopramide, lansoprazole, omeprazole, omeprazole sodium, omeprazole magnesium, rabeprazole sodium, pantoprazole sodium, domperidone, famotidine, ondansetron hydrochloride, tropisetron hydrochloride, irsogladine maleate, bisacodyl, loperamide hydrochloride, atropine sulfate, chlorpheniramine maleate, acetaminophen, ibuprofen, amantadine, procaterol hydrochloride, bambuterol hydrochloride, formoterol fumarate, clenbuterol hydrochloride, cetirizine hydrochloride, levocetirizine hydrochloride, bromhexine hydrochloride, cyproheptadine hydrochloride, salbutamol sulfate, terbutaline sulfate, loratadine, pholcodine, benproperine phosphate, aminophylline, doxofylline, ambroxol hydrochloride, penfluridol, methylcobalamin, cobamamide, diazepam, oxazepam, clonazepam, lorazepam, mirtazapine, olanzapine, aripiprazole, risperidone, zolmitriptan, phenobarbital, haloperidol, chlorpromazine hydrochloride, trihexyphenidyl hydrochloride, methylphenidate hydrochloride, atomoxetine hydrochloride, clomipramine hydrochloride, oxybutynin hydrochloride, zolpidem tartrate, chlorprothixene, codeine phosphate, donepezil hydrochloride, rivastigmine hydrogen tartrate, memantine hydrochloride, carbidopa, pramipexole hydrochloride, midazolam maleate, levothyroxine sodium, carbimazole, methimazole, stanozolol, dexamethasone, hydrocortisone, cydiodine, alfacalcidol, terazosin hydrochloride, desmopressin, pancreatic kininogenase, ubenimex, ebastine, leucovorin calcium, methotrexate, busulfan, semustine, leflunomide, temozolomide, primaquine phosphate, dihydroartemisinin, baclofen, adenine phosphate, stavudine, furazolidone, ribavirin, adefovir dipivoxil, oseltamivir phosphate, erythromycin cydocarbonate, erythromycin ethylsuccinate, berberine hydrochloride, folic acid, calcium pantothenate, calcium carbonate, calcium citrate, coenzyme Q10, vitamin B1, vitamin B2, vitamin B4, vitamin B6, vitamin C, vitamin D2, vitamin D3, and vitamin E.

The present disclosure aims to further provide a method for preparing the minitablet, including: mixing the active ingredient and/or the excipient to obtain the minitablet.

In some embodiments, the active ingredient is subjected to a pre-treatment, and the pre-treatment is preferably micronization.

In some embodiments, the method comprises:
(1) dispersing the active ingredient in a solvent, spraying a resulting dispersion into the filler, drying, and mixing evenly with the disintegrant, the lubricant, and/or the glidant to obtain a particle; and
(2) preparing the particle into the minitablet.

In some embodiments, in step (1), the drying is conducted by spray drying, and the drying is conducted at a temperature of 25° C. to 60° C., and preferably 25° C. to 40° C.

In some embodiments, in step (1), the spraying is conducted by using an atomization system with a droplet size D₉₀ of less than or equal to 500 μm, and preferably less than or equal to 100 μm.

In some embodiments, in step (1) or (2), the particle has a particle diameter D₉₀ of less than or equal to 250 μm, and preferably less than or equal to 150 μm.

The present disclosure further aims to provide a minitablet formulation, including the minitablet, where a number of the minitablet included in the formulation is single or multiple.

In some embodiments, a dosage form of the minitablet formulation is any one or more selected from the group consisting of a tablet, a buccal tablet, a sublingual tablet, an oral patch, an orally disintegrating tablet, a chewable tablet, a dispersible tablet, a soluble tablet, a fast dissolving tablet, an effervescent tablet, a vaginal tablet, a vaginal effervescent tablet, an enteric-coated tablet, an immediate-release tablet, a sustained-release tablet, a controlled-release tablet, and an implantable tablet.

Compared with the prior art, the present disclosure has the following advantages:
(1) In the present disclosure, the preparation method of the minitablet greatly reduces the dosage of excipients for some drugs by improving the types of excipients. The proportion of active ingredients can be increased to 40% to 80% according to the physical and chemical properties of different active ingredients. The preparation method effectively solves the problem of content uniformity while reducing the complexity and difficulty of the process, and is more suitable for industrial production.
(2) In the present disclosure, the prepared minitablet has the advantages that cannot be replaced by conventional tablets in clinical use: they can accurately control the dosage, thereby reducing various risks caused by inaccurate dosages, is small in size and can avoid the problem of swallowing difficulties in children and the elderly.

### DETAILED DESCRIPTION OF THE EMBODIMENTS

The present disclosure is further described below with reference to specific examples, in order to make the technical solutions of the present disclosure easier to understand and grasp, but the scope of the present disclosure is not limited thereto. The experimental methods described in the following examples are conventional methods unless otherwise specified; and the reagents and materials can be commercially available.

### Example 1

A minitablet consisted of the following components shown in the table below:

TABLE 1
Composition of Example 1

| | | Parts by |
|---|---|---|
| Component | Specific example | weight |
| Active | Levothyroxine sodium | 0.03 |
| ingredient | | |
| Filler | Mixture of microcrystalline | 25 |
| | cellulose, erythritol, xylan, | |
| | and corn starch in a mass | |
| | ratio of 3:1:1:2 | |
| Disintegrant | Mixture of soybean polysaccharide, | 6 |
| | glucuronic acid, and alginic acid | |
| | in a mass ratio of 1:1:1 | |
| Lubricant | Glyceryl distearate | 7 |

The method for preparing the minitablet was performed by the following steps:
(1) The active ingredient was micronized to a particle size of D₉₀=9.3 μm, dispersed in water, and sprayed into the filler using an atomization system with a droplet size of D₉₀ being 87.6 μm. The resulting mixture was subjected to spray drying at not greater than 40° C., and mixed evenly with the disintegrant and the lubricant to obtain particles having a particle size of D₉₀=143.1 μm.
(2) The particles were prepared into a minitablet having a diameter of 3 mm and a tablet weight of 20 mg.

### Example 2

A minitablet consisted of the following components shown in the table below:

TABLE 2
Composition of Example 2

| | | Parts by |
|---|---|---|
| Component | Specific example | weight |
| Active | Oseltamivir phosphate | 40 |
| ingredient | | |
| Filler | Mixture of microcrystalline | 30 |
| | cellulose, erythritol, xylan, | |
| | and wheat starch in a mass | |
| | ratio of 6:1:2:5 | |
| Disintegrant | Mixture of soybean polysaccharide, | 3 |
| | glucuronic acid, and alginic acid | |
| | in a mass ratio of 5:1:3 | |
| Glidant | Sodium stearyl fumarate | 4 |

The method for preparing the minitablet was performed by the following steps:
(1) The active ingredient was micronized to a particle size of D₉₀=43.6 μm, dispersed in water, and sprayed into the filler using an atomization system with a droplet size of D₉₀ being 378.5 μm. The resulting mixture was subjected to spray drying at not greater than 25° C., and mixed evenly with the disintegrant and the glidant to obtain particles having a particle size of D₉₀=223.7 μm.
(2) The particles were prepared into a minitablet having a diameter of 2 mm and a tablet weight of 10 mg.

### Example 3

A minitablet consisted of the following components shown in the table below:

TABLE 3
Composition of Example 3

| | | Parts by |
|---|---|---|
| Component | Specific example | weight |
| Active | Digoxin | 0.25 |
| ingredient | | |
| Filler | Mixture of microcrystalline cellulose, | 30 |
| | erythritol, xylan, and tapioca starch | |
| | in a mass ratio of 2:1:1:3 | |
| Disintegrant | Mixture of mannose and alginic acid | 8 |
| | in a mass ratio of 3:2 | |
| Lubricant | Magnesium aluminum silicate | 10 |

The method for preparing the minitablet was performed by the following steps:
(1) The active ingredient was micronized to a particle size of D₉₀=17.8 μm, dispersed in water, and sprayed into the filler using an atomization system with a droplet size of D₉₀ being 453.9 μm. The resulting mixture was subjected to spray drying at not greater than 60° C., and mixed evenly with the disintegrant and the lubricant to obtain particles having a particle size of D₉₀=101.6 μm.
(2) The particles were prepared into a minitablet having a diameter of 3 mm and a tablet weight of 30 mg.

### Example 4

A minitablet consisted of the following components shown in the table below:

TABLE 4
Composition of Example 4

| | | Parts by |
|---|---|---|
| Component | Specific example | weight |
| Active | Captopril | 35 |
| ingredient | | |
| Filler | Mixture of microcrystalline cellulose, | 22 |
| | erythritol, xylan, and lotus root starch | |
| | in a mass ratio of 3:1:2:2 | |
| Disintegrant | Mixture of tremella polysaccharide and | 8 |
| | alginic acid in a mass ratio of 2:3 | |
| Lubricant | Behenoyl polyoxyl-8 glycerides | 5 |

The method for preparing the minitablet was performed by the following steps:
(1) The active ingredient were mixed with the filler, disintegrant, and lubricant evenly to obtain particles having a particle size of D₉₀=206.3 μm.
(2) The particles were prepared into a minitablet having a diameter of 3 mm and a tablet weight of 25 mg.

### Example 5

A minitablet consisted of the following components shown in the table below:

TABLE 5
Composition of Example 5

| | | Parts by |
|---|---|---|
| Component | Specific example | weight |
| Active | Cetirizine hydrochloride | 46 |
| ingredient | | |
| Filler | Mixture of microcrystalline cellulose, | 25 |
| | erythritol, xylan, and corn starch in | |
| | a mass ratio of 6:1:1:5 | |
| Disintegrant | Mixture of soybean polysaccharide and | 6 |
| | alginic acid in a mass ratio of 6:1 | |
| Lubricant | Glyceryl distearate | 10 |
| Binder | Hypromellose | 5 |

The method for preparing the minitablet was performed by the following steps:
(1) The active ingredient was micronized to a particle size of D₉₀=36.9 μm, dispersed in water, and sprayed into the filler using an atomization system with a droplet size of D₉₀ being 92.7 μm. The resulting mixture was subjected to spray drying at not greater than 40° C., and mixed evenly with the disintegrant and the lubricant to obtain particles having a particle size of D₉₀=203.6 μm.
(2) The particles were mixed with the binder to obtain a minitablet having a diameter of 3 mm and a tablet weight of 30 mg.

### Example 6

A minitablet consisted of the following components shown in the table below:

TABLE 6
Composition of Example 6

| | | Parts by |
|---|---|---|
| Component | Specific example | weight |
| Active | Risperidone | 1.5 |
| ingredient | | |
| Filler | Mixture of microcrystalline cellulose, | 20 |
| | erythritol, xylan, and lotus root starch | |
| | in a mass ratio of 4:1:1:3 | |
| Disintegrant | Mixture of soybean polysaccharide and | 8 |
| | alginic acid in a mass ratio of 6:1 | |
| Lubricant | Glyceryl monostearate | 5 |
| Binder | Hydroxypropyl cellulose | 1 |

The method for preparing the minitablet was performed by the following steps:
(1) The active ingredient was micronized to a particle size of D₉₀=9.5 μm, dispersed in water, and sprayed into the filler using an atomization system with a droplet size of D₉₀ being 81.2 μm. The resulting mixture was subjected to spray drying at not greater than 40° C., and mixed evenly with the disintegrant and the lubricant to obtain particles having a particle size of D₉₀=112.6 μm.
(2) The particles were mixed with the binder to obtain a minitablet having a diameter of 3 mm and a tablet weight of 20 mg.

### Example 7

This example differed from Example 1 in that the filler was different.

A minitablet consisted of the following components shown in the table below:

TABLE 7
Composition of Example 7

| | | Parts by |
|---|---|---|
| Component | Specific example | weight |
| Active | Levothyroxine sodium | 0.03 |
| ingredient | | |
| Filler | Mixture of mannitol and pregelatinized | 25 |
| | starch in a mass ratio of 1:1 | |
| Disintegrant | Mixture of soybean polysaccharide, | 6 |
| | glucuronic acid, and alginic acid | |
| | in a mass ratio of 1:1:1 | |
| Lubricant | Glyceryl distearate | 7 |

The method for preparing the minitablet was performed by the following steps:
(1) The active ingredient was micronized to a particle size of D₉₀=9.3 μm, dispersed in water, and sprayed into the filler using an atomization system with a droplet size of D₉₀ being 87.6 μm. The resulting mixture was subjected to spray drying at not greater than 40° C., and mixed evenly with the disintegrant and the lubricant to obtain particles having a particle size of D₉₀=143.1 μm.
(2) The particles were prepared into a minitablet having a diameter of 3 mm and a tablet weight of 20 mg.

### Example 8

This example differed from Example 1 in that the disintegrant was different.

A minitablet consisted of the following components shown in the table below:

TABLE 8
Composition of Example 8

| | | Parts by |
|---|---|---|
| Component | Specific example | weight |
| Active | Levothyroxine sodium | 0.03 |
| ingredient | | |
| Filler | Mixture of microcrystalline cellulose, | 25 |
| | erythritol, xylan, and corn starch | |
| | in a mass ratio of 3:1:1:2 | |
| Disintegrant | Croscarmellose sodium | 6 |
| Lubricant | Glyceryl distearate | 7 |

The method for preparing the minitablet was performed by the following steps:
(1) The active ingredient was micronized to a particle size of D₉₀=9.3 μm, dispersed in water, and sprayed into the filler using an atomization system with a droplet size of D₉₀ being 87.6 μm. The resulting mixture was subjected to spray drying at not greater than 40° C., and mixed evenly with the disintegrant and the lubricant to obtain particles having a particle size of D₉₀=143.1 μm.
(2) The particles were prepared into a minitablet having a diameter of 3 mm and a tablet weight of 20 mg.

### Example 9

This example differed from Example 1 in that the disintegrant and filler were different.

A minitablet consisted of the following components shown in the table below:

TABLE 9
Composition of Example 9

| | | Parts by |
|---|---|---|
| Component | Specific example | weight |
| Active | Levothyroxine sodium | 0.03 |
| ingredient | | |
| Filler | Microcrystalline cellulose | 25 |
| Disintegrant | Croscarmellose sodium | 6 |
| Lubricant | Glyceryl distearate | 7 |

The method for preparing the minitablet was performed by the following steps:
(1) The active ingredient was micronized to a particle size of D₉₀=9.3 μm, dispersed in water, and sprayed into the filler using an atomization system with a droplet size of D₉₀ being 87.6 μm. The resulting mixture was subjected to spray drying at not greater than 40° C., and mixed evenly with the disintegrant and the lubricant to obtain particles having a particle size of D₉₀=143.1 μm.
(2) The particles were prepared into a minitablet having a diameter of 3 mm and a tablet weight of 20 mg.

### Example 10

This example differed from Example 4 in that the filler was different.

A minitablet consisted of the following components shown in the table below:

TABLE 10
Composition of Example 10

| | | Parts by |
|---|---|---|
| Component | Specific example | weight |
| Active ingredient | Captopril | 35 |
| Filler | Lactose monohydrate | 22 |
| Disintegrant | Mixture of tremella polysaccharide | 8 |
| | and alginic acid in a mass ratio of 2:3 | |
| Lubricant | Behenoyl polyoxyl-8 glycerides | 5 |

The minitablet was prepared by the same process as that in Example 4.

### Example 11

This example differed from Example 5 in that the filler was different.

A minitablet consisted of the following components shown in the table below:

TABLE 11
Composition of Example 11

| | | Parts by |
|---|---|---|
| Component | Specific example | weight |
| Active | Cetirizine hydrochloride | 46 |
| ingredient | | |
| Filler | Mixture of microcrystalline cellulose, | 25 |
| | erythritol, xylan, and corn starch in | |
| | a mass ratio of 6:1:1:5 | |
| Disintegrant | Crospovidone | 6 |
| Lubricant | Glyceryl distearate | 10 |
| Binder | Hypromellose | 5 |

The minitablet was prepared by the same process as that in Example 4.

### Example 12

This example differed from Example 6 in that the filler was different.

A minitablet consisted of the following components shown in the table below:

TABLE 12
Composition of Example 12

| | | Parts by |
|---|---|---|
| Component | Specific example | weight |
| Active | Risperidone | 1.5 |
| ingredient | | |
| Filler | calcium hydrogen phosphate | 20 |
| Disintegrant | Mixture of soybean polysaccharide | 8 |
| | and alginic acid in a mass ratio of 6:1 | |
| Lubricant | Glyceryl monostearate | 5 |
| Binder | Hydroxypropyl cellulose | 1 |

The minitablet was prepared by the same process as that in Example 6.

### Comparative Example 1

This comparative example differed from Example 1 in that the particle size of the active ingredient was different.

A minitablet consisted of the same components as those in Example 1.

The method for preparing the minitablet was performed by the following steps:
(1) The active ingredient was micronized to a particle size of D₉₀=312.6 μm, dispersed in water, and sprayed into the filler using an atomization system with a droplet size of D₉₀ being 87.6 μm. The resulting mixture was subjected to spray drying at not greater than 40° C., and mixed evenly with the disintegrant and the lubricant to obtain particles having a particle size of D₉₀=143.1 μm.
(2) The particles were prepared into a minitablet having a diameter of 3 mm and a tablet weight of 20 mg.

### Comparative Example 2

This comparative example differed from Example 2 in that the particle size of the particle was different.

A minitablet consisted of the same components as those in Example 2.

The method for preparing the minitablet was performed by the following steps:
(1) The active ingredient was micronized to a particle size of D₉₀=43.6 μm, dispersed in water, and sprayed into the filler using an atomization system with a droplet size of D₉₀ being 378.5 μm. The resulting mixture was subjected to spray drying at not greater than 25° C., and mixed evenly with the disintegrant and the glidant to obtain particles having a particle size of D₉₀=326.8 μm.
(2) The particles were prepared into a minitablet having a diameter of 2 mm and a tablet weight of 10 mg.

### Comparative Example 3

This comparative example differed from Example 3 in that the particle size of the droplet was different.

A minitablet consisted of the same components as those in Example 3.

The method for preparing the minitablet was performed by the following steps:
(1) The active ingredient was micronized to a particle size of D₉₀=9.3 μm, dispersed in water, and sprayed into the filler using an atomization system with a droplet size of D₉₀ being 631.2 μm. The resulting mixture was subjected to spray drying at not greater than 40° C., and mixed evenly with the disintegrant and the lubricant to obtain particles having a particle size of D₉₀=143.1 μm.
(2) The particles were prepared into a minitablet having a diameter of 3 mm and a tablet weight of 20 mg.

### Evaluation on Effect

### 1. Weight Variation Test

The test was based on the weight variations of tablets (General Chapter 0101) in the Part IV of the 2020 version “Chinese Pharmacopoeia”.

20 minitablets prepared in each example or comparative example were taken, and a total weight was accurately weighed. After obtaining an average tablet weight, each tablet was accurately weighed separately. The weight of each tablet was compared with the average tablet weight. According to the provisions in Table 13, no more than 2 tablets might exceed a weight variation limit, and no one tablet might exceed the limit by one time.

TABLE 13
Weight variation limit provisions

| Average tablet weight or | Weight variation |
|---|---|
| expressed tablet weight | limit |
| less than or equal to 0.30 g | ±7.5% |
| 0.30 g and greater than 0.30 g | ±5% |

According to the above method, the minitablets prepared in Examples 1 to 12 and Comparative Examples 1 to 3 were inspected for weight variations. The results are shown in Table 14.

TABLE 14
Weight variation effect data

| Experimental group | Tablet weight variation |
|---|---|
| Example 1 | +6.4%; −5.9% |
| Example 2 | +6.3%; −6.8% |
| Example 3 | +5.9%; −5.0% |
| Example 4 | +6.3%; −6.7% |
| Example 5 | +6.4%; −6.7% |
| Example 6 | +5.7%; −6.1% |
| Example 7 | +5.5%; −6.0% |
| Example 8 | +7.0%; −7.8% |
| Example 9 | +6.6%; −7.1% |
| Example 10 | +8.0%; −7.6% |
| Example 11 | +7.6%; −8.3% |
| Example 12 | +8.5%; −7.9% |
| Comparative Example 1 | +8.1%; −7.6% |
| Comparative Example 2 | +9.4%; −8.8% |
| Comparative Example 3 | +7.9%; −8.3% |

As shown in the above table, the weight variation of the minitablets provided in the present disclosure is small, indicating that the preparation method in the present disclosure shows high stability.

### 2. Content Uniformity Test

The test was in accordance with the content uniformity inspection method (General Chapter 0941) in Part IV of the 2020 edition “Chinese Pharmacopoeia”.

Unless otherwise specified, 10 tested samples were selected, and a relative content xi of each single dose with a labeled amount as 100 was measured according to the method specified under each variety, and a mean X̅ and a standard deviation S, as well as an absolute value A (A=|100−X̅|) of the difference between the labeled amount and the mean were calculated.

If A+2.2S≤L, the content uniformity of the test sample complied with the provisions;
if A+S>L, the content uniformity of the test sample did not meet the provisions;
if A+2.2S>L and A+S≤L, 20 additional tested samples should be taken for retesting.

In the above formula, L was a specified value, unless otherwise specified, L=15.0.

Referring to the above method, the content uniformity was measured on the minitablets prepared in Examples 1 to 12 and Comparative Examples 1 to 3. The results are shown in Table 15.

TABLE 15
Content uniformity

| Experimental group | Content uniformity |
|---|---|
| Example 1 | 6.9 |
| Example 2 | 6.6 |
| Example 3 | 7.3 |
| Example 4 | 8.1 |
| Example 5 | 8.2 |
| Example 6 | 8.9 |
| Example 7 | 9.5 |
| Example 8 | 10.1 |
| Example 9 | 12.4 |
| Example 10 | 14.6 |
| Example 11 | 13.6 |
| Example 12 | 12.7 |
| Comparative Example 1 | 19.3 |
| Comparative Example 2 | 18.1 |
| Comparative Example 3 | 16.9 |

As shown in the above table, the content uniformity of the minitablets provided by the present disclosure meets the provisions and is far less than the regulatory requirements, and the minitablets show desirable content uniformity.

### 3. Dissolution Rate Test

The test was conducted with reference to the dissolution methods under each drug item in Part II of the 2020 edition “Chinese Pharmacopoeia”.

The dissolution rates of the minitablets prepared in Examples 1 to 12 and Comparative Examples 1 to 3 were measured according to the above method.

TABLE 16
Dissolution rate data

| Experimental group | Dissolution rate (%) |
|---|---|
| Example 1 | 96.5 |
| Example 2 | 95.2 |
| Example 3 | 95.5 |
| Example 4 | 93.6 |
| Example 5 | 92.8 |
| Example 6 | 93.1 |
| Example 7 | 90.4 |
| Example 8 | 89.1 |
| Example 9 | 87.5 |
| Example 10 | 84.3 |
| Example 11 | 79.6 |
| Example 12 | 85.3 |
| Comparative Example 1 | 80.2 |
| Comparative Example 2 | 83.6 |
| Comparative Example 3 | 81.9 |

As shown in the above table, the minitablets provided by the present disclosure have relatively high dissolution rates and can be quickly disintegrated and fully released after taking, which is conducive to better absorption in vivo, thereby improving bioavailability.

### 4. Stability Test

The relevant substances in the minitablets of Examples 1 to 12 and Comparative Examples 1 to 3 were detected with reference to the detection methods of relevant substances under each drug item in Part II of the “Chinese Pharmacopoeia”.

Test conditions: samples were placed under high temperature (40° C.), high humidity (RH75%), and light (5,000 lx) for 10 d and under acceleration (40° C.-RH75%) for 1 month, and the relevant substances were tested. The results are shown in the table below.

TABLE 17
Stability data

| | Total impurities (%) | | | | |
|---|---|---|---|---|---|
| Experimental | | 60° C.- | RH75%- | 5000 1x- | Acceleration- |
| group | 0 d | 10 d | 10 d | 10 d | 1 month |
| Example 1 | 1.019 | 1.135 | 1.101 | 1.123 | 2.021 |
| Comparative | 1.490 | 2.226 | 1.620 | 1.695 | 3.062 |
| Example 1 | | | | | |
| Example 2 | 0.128 | 0.146 | 0.162 | 0.282 | 0.373 |
| Comparative | 0.143 | 0.182 | 0.213 | 0.369 | 0.516 |
| Example 2 | | | | | |
| Example 3 | 1.052 | 1.203 | 1.160 | 1.049 | 1.303 |
| Comparative | 1.263 | 1.619 | 1.703 | 1.305 | 2.051 |
| Example 3 | | | | | |
| Example 4 | 0.512 | 0.762 | 0.659 | 0.692 | 0.967 |
| Example 5 | 0.672 | 0.792 | 0.721 | 0.862 | 1.036 |
| Example 6 | 0.126 | 0.192 | 0.139 | 0.129 | 0.375 |
| Example 7 | 1.050 | 1.251 | 1.233 | 1.306 | 2.450 |
| Example 8 | 1.069 | 1.302 | 1.161 | 1.473 | 2.336 |
| Example 9 | 1.395 | 1.690 | 1.765 | 1.609 | 2.906 |
| Example 10 | 0.603 | 0.862 | 0.903 | 0.933 | 1.406 |
| Example 11 | 0.669 | 0.902 | 0.936 | 0.996 | 1.631 |
| Example 12 | 0.130 | 0.306 | 0.290 | 0.143 | 0.709 |

As shown in the above table, the minitablets provided by the present disclosure have significant drug stability, and the composition of the excipients as well as the preparation method of the drug have a greater impact on the drug stability.

The above detailed description is specific to one of the possible embodiments of the present disclosure. The embodiments are not intended to limit the scope of the present disclosure. Any equivalent implementation or modification without departing from the present disclosure should be included within the scope of technical solutions of the present disclosure.

1. A minitablet, comprising the following components:
an active ingredient and/or an excipient, wherein
the minitablet has a diameter of 1 mm to 5 mm and a tablet weight of 1 mg to 50 mg; and
the excipient comprises any one or more selected from the group consisting of a filler, a disintegrant, a binder, a lubricant, and a glidant.

2. The minitablet of claim 1, wherein the minitablet has a diameter of 1 mm to 3 mm and a tablet weight of 2 mg to 30 mg.

3. The minitablet of claim 1, wherein the active ingredient has a particle size D₉₀ of less than or equal to 250 μm.

4. The minitablet of claim 1, wherein a mixture of the active ingredient and/or the excipient has a particle size D₉₀ of less than or equal to 350 μm.

5. The minitablet of claim 1, wherein the filler comprises any one or more selected from the group consisting of starch, pregelatinized starch, powdered sugar, lactose monohydrate, lactitol, sucrose, glucose, fructose, dextrin, cyclodextrin, powdered cellulose, microcrystalline cellulose, aerosil, mannitol, sorbitol, xylitol, erythritol, xylan, maltitol, glycine, calcium chloride, calcium sulfate, calcium carbonate, calcium phosphate, calcium hydrogen phosphate, calcium glycerophosphate, calcium carboxymethylcellulose, sodium chloride, sodium starch phosphate, aluminum chloride, aluminum hydroxide, aluminum silicate, calcium aluminum silicate, and magnesium carbonate;
the disintegrant comprises any one or more selected from the group consisting of starch, pregelatinized starch, polacrilin potassium, soybean polysaccharide, microcrystalline cellulose, sodium bicarbonate, potassium bicarbonate, potassium carbonate, citric acid, alginic acid, sodium alginate, sodium starch glycolate, methylcellulose, low-substituted hydroxypropyl cellulose, crospovidone, croscarmellose sodium, carboxymethylcellulose sodium, and carboxymethylcellulose calcium;
the binder comprises any one or more selected from the group consisting of gelatin, acacia, xanthan gum, tragacanth, polyethylene glycol, pregelatinized starch, polyvinyl alcohol, starch, dextrin, chitin, lactose monohydrate, sucrose, chitosan, glucose, dextran, copovidone, polyvinylpyrrolidone, carbomer, hydroxypropyl cellulose, methylcellulose, ethylcellulose, ethyl methylcellulose, hypromellose, low-substituted hypromellose, and carboxymethylcellulose sodium; and
the lubricant and/or the glidant comprises any one or more selected from the group consisting of silica, aerosil, talc, glyceryl behenate, glyceryl monostearate, glyceryl distearate, sucrose fatty acid ester, sucrose monolaurate, sucrose monopalmitate, polyethylene glycol, sodium laurylsulfate, docusate sodium, sodium palmitate, magnesium silicate, magnesium aluminum silicate, stearic acid, sodium stearate, calcium stearate, zinc stearate, magnesium stearate, and sodium stearyl fumarate.

6. The minitablet of claim 1, wherein the disintegrant is a mixture of a polysaccharide and the alginic acid;
a mass ratio of the polysaccharide to the alginic acid in the disintegrant is in a range of (2-6):(1-3); and
the polysaccharide is any one or more selected from the group consisting of fucose, mannose, soybean polysaccharide, black soybean polysaccharide, tremella polysaccharide, galactan, and glucuronic acid.

7. (canceled)

8. (canceled)

9. The minitablet of claim 1, wherein the disintegrant is a mixture of soybean polysaccharide, glucuronic acid, and alginic acid, wherein a mass ratio of the soybean polysaccharide, the glucuronic acid, and the alginic acid is in a range of (1-5):1:(1-3).

10. The minitablet of claim 1, wherein the disintegrant is a mixture of croscarmellose sodium and low-substituted hydroxypropyl cellulose and/or a mixture of sodium starch glycolate and pregelatinized starch;
a mass ratio of the croscarmellose sodium to the low-substituted hydroxypropyl cellulose in the mixture is in a range of (1-4):1; and
a mass ratio of the sodium starch glycolate to the pregelatinized starch in the mixture is in a range of (1-4):1.

11. (canceled)

12. The minitablet of claim 1, wherein the filler is a mixture of microcrystalline cellulose, erythritol, xylan, and starch;
a mass ratio of the microcrystalline cellulose, the erythritol, the xylan, and the starch in the filler is in a range of (3-6):1:(1-2):(2-5); and
the starch is any one or more selected from the group consisting of wheat starch, sweet potato starch, lotus root starch, corn starch, and tapioca.

13. (canceled)

14. (canceled)

15. The minitablet of claim 1, wherein the filler is a mixture of microcrystalline cellulose and mannitol and/or a mixture of the microcrystalline cellulose and lactose monohydrate;
a mass ratio of the microcrystalline cellulose to the mannitol in the mixture is in a range of (1-4):1; and
a mass ratio of the microcrystalline cellulose to the lactose monohydrate in the mixture is in a range of (1-4):1.

16. (canceled)

17. The minitablet of claim 1, comprising the filler and the disintegrant, wherein a mass ratio of the filler to the disintegrant is in a range of (1-2):(2-5).

18. The minitablet of claim 1, comprising the following components in parts by weight: 0.03 parts to 80 parts of the active ingredient, 20 parts to 30 parts of the filler, and 3 parts to 8 parts of the disintegrant.

19. The minitablet of claim 1, wherein the active ingredient is any one or more selected from the group consisting of:
captopril, indapamide, propranolol hydrochloride, bisoprolol fumarate, atenolol, bumetanide, furosemide, torasemide, minoxidil, bendroflumethiazide, hydrochlorothiazide, reserpine, digoxin, medigoxin, nifedipine, isosorbide dinitrate, isosorbide mononitrate, pentaerythritol tetranitrate, nitroglycerin, clonidine hydrochloride, warfarin sodium, spironolactone, lofexidine hydrochloride, glyburide, glimepiride, glipizide, voglibose, acarbose, clopidogrel bisulfate, aspirin, scopolamine butylbromide, anisodamine hydrobromide, metoclopramide, lansoprazole, omeprazole, omeprazole sodium, omeprazole magnesium, rabeprazole sodium, pantoprazole sodium, domperidone, famotidine, ondansetron hydrochloride, tropisetron hydrochloride, irsogladine maleate, bisacodyl, loperamide hydrochloride, atropine sulfate, chlorpheniramine maleate, acetaminophen, ibuprofen, amantadine, procaterol hydrochloride, bambuterol hydrochloride, formoterol fumarate, clenbuterol hydrochloride, cetirizine hydrochloride, levocetirizine hydrochloride, bromhexine hydrochloride, cyproheptadine hydrochloride, salbutamol sulfate, terbutaline sulfate, loratadine, pholcodine, benproperine phosphate, aminophylline, doxofylline, ambroxol hydrochloride, penfluridol, methylcobalamin, cobamamide, diazepam, oxazepam, clonazepam, lorazepam, mirtazapine, olanzapine, aripiprazole, risperidone, zolmitriptan, phenobarbital, haloperidol, chlorpromazine hydrochloride, trihexyphenidyl hydrochloride, methylphenidate hydrochloride, atomoxetine hydrochloride, clomipramine hydrochloride, oxybutynin hydrochloride, zolpidem tartrate, chlorprothixene, codeine phosphate, donepezil hydrochloride, rivastigmine hydrogen tartrate, memantine hydrochloride, carbidopa, pramipexole hydrochloride, midazolam maleate, levothyroxine sodium, carbimazole, methimazole, stanozolol, dexamethasone, hydrocortisone, cydiodine, alfacalcidol, terazosin hydrochloride, desmopressin, pancreatic kininogenase, ubenimex, ebastine, leucovorin calcium, methotrexate, busulfan, semustine, leflunomide, temozolomide, primaquine phosphate, dihydroartemisinin, baclofen, adenine phosphate, stavudine, furazolidone, ribavirin, adefovir dipivoxil, oseltamivir phosphate, erythromycin cydocarbonate, erythromycin ethylsuccinate, berberine hydrochloride, folic acid, calcium pantothenate, calcium carbonate, calcium citrate, coenzyme Q10, vitamin B1, vitamin B2, vitamin B4, vitamin B6, vitamin C, vitamin D2, vitamin D3, and vitamin E.

20. A method for preparing the minitablet of claim 1, comprising: mixing the active ingredient and/or the excipient to obtain the minitablet.

21. The method for preparing the minitablet of claim 20, wherein the active ingredient is subjected to a pre-treatment, and the pre-treatment is micronization.

22. The method for preparing the minitablet of claim 20, wherein the method comprises:
(1) dispersing the active ingredient in a solvent, spraying a resulting dispersion into the filler, drying, and mixing with the disintegrant, the lubricant, and/or the glidant to obtain a particle; and
(2) preparing the particle into the minitablet.

23. The method for preparing the minitablet of claim 22, wherein in step (1), the drying is conducted by spray drying, and the drying is conducted at a temperature of 25° C. to 60° C.

24. The method for preparing the minitablet of claim 22, wherein in step (1), the spraying is conducted by using an atomization system, with a droplet size D₉₀ of less than or equal to 500 μm.

25. A minitablet formulation, comprising the minitablet of claim 1, wherein a number of the minitablet comprised in the formulation is single or multiple.

26. The minitablet formulation of claim 25, wherein a dosage form of the minitablet formulation is any one or more selected from the group consisting of a tablet, a buccal tablet, a sublingual tablet, an oral patch, an orally disintegrating tablet, a chewable tablet, a dispersible tablet, a soluble tablet, a fast dissolving tablet, an effervescent tablet, a vaginal tablet, a vaginal effervescent tablet, an enteric-coated tablet, an immediate-release tablet, a sustained-release tablet, a controlled-release tablet, and an implantable tablet.
